(19) United States
(12) Patent Application Publication
(10) Pub. No.: US 2004/0185129 A1
(43) Pub. Date: Sep. 23, 2004

(54) Salvia hispanica l (chia) in the management and treatment of cardiovascular disease diabetes and asociated risk factors

(76) Inventor: Vladimir Vuksan, Toronto (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 10/469,538

(22) PCT Filed: Mar. 11, 2002
(86) PCT No.: PCT/CA02/00327

Related U.S. Application Data
(60) Provisional application No. 60/274,256, filed on Mar. 9, 2001.

(51) Int. Cl. A61K 35/78

(52) U.S. Cl. 424/776

(57) ABSTRACT

Described is use of Salvia hispanica L. (Chia) for controlling, in one embodiment reducing, blood glucose levels, preferably post-prandial blood glucose levels. This is useful in both non-diabetic and diabetic individuals, but especially in diabetic individuals. Also described is the use of chia in reducing postprandial blood glucose, insulin sensitivity, blood pressure, and oxidative stress in such individuals. The present invention further found that Chia can be used to improve endothelial function, coagulation, fibrinolysis and iron status. The present invention further encompasses the use of Chia in the treatment and/or management of diabetes and/or the treatment and management of diabetes associated conditions or risk factors, such as one or more of the following: blood pressure and blood glucose levels, post-prandial glycemia, inflammatory factors (C-reactive protein), coagulation (fibrinogen, factor VIII, von Willenbrand factor), and fibronolytic factors (such as t-PA), iron status and endothelial function, (such as increase in nitric oxide generation). In one embodiment the invention relates to dietary approaches to such treatment and management.

### RELATED APPLICATION

This application claims priority from U.S. Patent Application No. 60/274,256, filed Mar. 9, 2001, which is incorporated herein by reference. As Mar. 9, 2002 falls on a Saturday, this application is being filed on the next available business day, Monday Mar. 11, 2002, in accordance with Article 4 of the Stockholm Act of the Paris Convention for the Protection of Industrial Property and Article 18 of the Patent Cooperation Treaty.

### FIELD OF THE INVENTION

This invention relates to the field of the treatment and/or management of diabetes and/or the treatment and management of diabetes and/or cardiovascular disease associated conditions or risk factors, such as one or more of the following: blood pressure, blood glucose levels, post-prandial glycemia, inflammatory factors (C-reactive protein), coagulation (fibrinogen, factor VIII), fibrinolytic factors such as t-PA, iron status and endothelial function. In one embodiment the invention relates to dietary approaches to such treatment and management and to related methods and uses of chia and to the compositions for effecting the and methods and uses of the invention.

### BACKGROUND OF THE INVENTION

Diabetes, Coronary Heart Disease and Associated Factors

Abnormal glucose tolerance and insulin resistance associated with diabetes is related to multiple cardiovascular risk factors that especially reduce HDL, elevated serum triglycerides and hypertension (Liese et al. (1998). Other important risk factors associated with diabetes include endothelial dysfunction, inflammation factor, coagulation (fibrinogen, factor VIII, vonWillebrand factor) and fibrinolysis. When clustered in type 2 diabetes, these abnormalities accelerate the process of arteriosclerosis and increase the risk of coronary heart disease (CHD) morbidity and mortality, (Trevisan et al. 1998, Epstein et al 2000). The majority of type 2 diabetic individuals develop most of these metabolic abnormalities in relation to development of disease and/or its progression.

Hyperglycemia and diabetes are strong and independent risk factors of both all-cause and cardiovascular (CVD) mortality (Wing et al. (1998). These links are more pronounced when the diabetes is associated with other unfavourable risk factors such as hyperlipidemia (Goldsmith et al. (1994)), hypertension (Burt et al. (1995), or a cluster of metabolic disorders (Stamler et al. (1993)). Since people with diabetes have almost twice the risk of dying from CVD (69.6%) compared to people in the general U.S. population (Gu et al. (1998), the control of high glucose levels and other concomitant coronary heart disease (CHD) risk factors represents the most effective approach to prevention (Savage (1996). Most recent studies suggest that an effective treatment of type 2 diabetes lies beyond glycemic control, and that other therapeutic strategies may be involved (UKPDS 49, Lancet 2000). Some of the most common abnormalities associated with diabetes include endothelial dysfunction, inflammation, and problems with fibrinolysis, platelet aggregation and blood coagulation. Each of these abnormalities, and especially when occurring together plays a major role in the pathogenesis of athero-thrombosis.

Prospective and case-control studies have indicated that many of the proteins involved in coagulation and fibrinolysis that might contribute to a thrombotic tendency are in fact related to the development of heart disease, with much higher risk being in individuals with diabetes. The suppression of fibrinolysis due to high plasminogen-activator inhibitor (PAI-I) and increased plasma concentration of factor VIII and von Willebrand factor are associated with increased development of myocardial infarction (MI). In additional, high concentration of tissue plasminogen activator (t-PA) also increase MI (Thompson 1995). PAI-I is inhibitor of plasminogen activation and it is produced in endothelium, but is also present in platelets and is considered to be an important regulator of fibrinolysis (Epstein et al. 2000). Inflammation also plays a key role in the pathogenesis of thrombosis, and measurements of high-sensitivity C-reactive protein (CRP)— a sensitive marker for systematic inflammation-can identify individuals at high risk of developing CHD (Ridker et al. 2000).

The importance of stronger nutrition-hygienic measures has been stressed repeatedly for the public at large (Stamler et al. (1993); National Cholesterol Education Program: Second report of the expert panel on detection, evaluation, and treatment of high blood cholesterol in adults (adult treatment panel II). Circulation. 1994; 89:1333-1445)). When these measures prove inadequate, an aggressive drug therapy is often required to meet the conventional treatment guidelines (National Cholesterol Education Program: Second report of the expert panel on detection, evaluation, and treatment of high blood cholesterol in adults (adult treatment panel II). Circulation. 1994; 89:1333-1445)). In the general population, this approach has been shown to be effective in lowering both the prevalence of hypertension (Burt et al. (1995), and serum cholesterol levels (Johnson et al. (1993)), but has not reduced the incidence of diabetes (Harris et al. (1998). Two most recent population intervention studies conducted in Finland (Tuomilehto NEJM, 2001) and USA (www.niddk.nih.gov/8_{—}8_{—}01.htm) indicate that healthy diet; modest reduction in body weight and increase in physical activities can reduce number of new cases in diabetes for nearly 60 percent.

Although it has been extensively described by Epstein et al. (2000), Liese et al. (1998); Trevisan et al. (1998; Himswarth (1936); Haffner et al. (1986); Helmrich et al. (1994)), followed-up (Reaven (1994)), and had its high prevalence determined, no specific recommendations for treatment of diabetes related risk factor cluster of conventional (glucose, lipids, hypertension) and emerging risk factors (fibrinolysis, coagulation and inflammation) in type 2 diabetes have been proposed by medical society or health agencies. In practice, initial therapy of individual risk factors such as moderate dyslipidemia, hypertension or hyperglycemia is nonpharmacological. Treatment will often include behavioral changes to reduce body weight, increase physical activity, and moderate alcohol consumption. To achieve nutritional goals, there are three main approaches: a high-carbohydrate/low-fat diet (National Cholesterol Education Program: Second report of the expert panel on detection, evaluation, and treatment of high blood cholesterol in adults (adult treatment panel II) Circulation 89:1333-1445 (1994)), sharing calories between monounsaturated fat and complex carbohydrate at the expense of saturated fat (American Diabetes Association (ADA): Nutrition Recommendations and principles for people with diabetes mellitus. Diabetes Care 22:s42-s43 (1999)), or supplementing a high-carbohydrate/low-fat diet with exercise (Stefanick et al. (1998)). Except weight loss for reduction of inflammation, no dietary therapies have been recommended to improve coagulation or fibrinolysis.

Tighter fasting and postprandial glycemic control results in a considerable reduction in CHD and all-cause mortality (Wei et al. (1998)), as well as fewer long-term microvascular complications both in type 1 (DCCT Research Group: The effect of intensive treatment of diabetes on the development and progression of long-term complications in insulin-dependent diabetes mellitus. The diabetes control and complications trial. New Engl J Med 329:977-986 (1993) and type 2 diabetes (UK Prospective Diabetes Study (UKPDS) Group: Effect of intensive blood-glucose control with metformin on complications in overweight patients with type 2 diabetes: UKPDS 34. Lancet 352:854-865, 1998).

There is a need for better treatment and management of diabetes, preferably Type 2 Diabetes, and of Cardiovascular heart disease and associated factors. Preferably the treatment and management is in the form of dietary or dietary supplement and/or related therapy.

Role of Omega-3 Fatty Acid in Diabetes and Cardiovascular Disease and Treatment or Management Thereof

Although there is no convincing evidence that omega-3 fatty acids play an important role in diabetes or cardiovascular disease, more recently there are some indications in cardiprotective function of omega-3 fatty acids. The potential role of fish oil in cardiovascular disease risk reduction first came from early observations involving Inuits in Greenland, who despite 40% of calories from fat (mainly from marine source) had lower incidence of CHD (Mouratoff et al. 1967). Also, large prospective study “GISSI-Prevenzione” conducted in over 11,000 MI survival patients demonstrated significant reduction of CHD death for 17% (GISSI_Prevenzione Investigators. 1999). Consumption of fish oil in meta analysis studies have shown reduction type 2 diabetes in significant lowering of serum triglycerides (Montori et al. 2000). Based on recent population studies from Harvard School of Medicine conducted in health professional and nurses, diets rich in omega-3 fat have been shown to have a protective role in preventing heart disease. In another secondary prevention study a group of authors followed group of individuals for 27 months and found that supplementation of margarine high in plant source of omega 3 added to Mediterranean diet reduced re-occurrence of MI for 58% (de Lorgeril et al 1998). This, so called Lyon Diet Heart Study stimulate ad great number of new studies in this area, also our interest in studying the effect of plant source of omega-3 fatty acids in clinical setting in type 2 diabetes. The results of these studies are shown in Table 1.

Previously other authors studied the effect of plant source of omega-3 fatty acids by feeding flax seed added to test meal to healthy volunteers and found decrease in postprandial plasma glucose excursions (Wolever et al. (1995); Jenkins et al. (1995). The mechanism is presumed to involve slowing carbohydrate absorption (Wolever et al. (1995)) that is most likely due to the soluble fiber and other flaxseed components of flaxseed. In the case of clinical studies however, in the case of flaxseed, increase the viscosity of digesta in the human gut that reduce postprandial blood glucose (Wolever 1995). In a long term study in which ground flax seed were added to study muffins authors have seen reduction in serum lipids (Cunnane 1995)).

Chia (Saliva Hispanica L.

Chia or Salvia Hispanica is an estival growing annual species belonging to the family Labiata that is indigenous to Central and South America, particularly the Rocky Mountains area extending from the Mexican western central area towards northern Guatemala. A sample of references on chia can be found in the list of references provided herein.

Pre-Columbian civilizations, mainly Aztecs, used chia as a raw material for a number of applications, such as in a variety of medicinal and nutritional compounds, and in substances such as paints. Chia was extremely important to Pre-Columbian societies. From the point of view of significance, only corn and beans surpassed it.

Although chia was originally part of the South and Central American and U.S. Southwest indigenous diet, this changed with colonization and modernization. Today, Mexican Indian descendants still grow chia on a small scale using rudimentary technological methods, for preparing a popular beverage called “Chia fresca”.

Chia is also grown today for use as an invaluable binder in industrial compounds, such as varnish, paints and cosmetics.

There is a need to study and determine the nutritional and medicinal benefits of chia. A better understanding of the effects of chia, may lead to new uses of chia, the development of a better dietary regime or new pharmaceutical or other compositions for the treatment or control of a number of medical conditions or other applications.

### SUMMARY OF THE INVENTION

The present inventor has determined that the addition of seeds Salvia Hispanica L., (Chia) consumed alone or incorporated into the food to a diet of an animal enhances conventional treatment outcomes, assessed primarily by blood glucose, insulin, insulin sensitivity, diastolic and systolic blood pressure, and secondarily inflammation, coagulation, fibrinolysis and endothelial function.

Accordingly, in one aspect the present invention provides a sufficient or effective amount of Chia seeds (e.g. whole, ground, liquefied, an extract or as part of a chia seed composition) which when given to an animal, preferably at an appropriate time, reduces fasting and postprandial blood glucose in the animal,

Preferably, chia seed and/or a chia seed composition according to the invention is consumed on its own, or formulated into a liquid; powder or formulated as part of a food.

According to another aspect the present invention provides a method for treating, controlling, managing, preferably reducing, risk factors for heart disease including those risk factors selected from the group consisting of: blood pressure, inflammation (CRP), coagulation (fabrinogen, factor VIII and von Willbrand factor), coagulation (e.g. by increasing t-PA) in an animal comprising administering to the animal a sufficient or effective amount of Chia seed (e.g. whole, ground, liquefied, an extract or as part of a chia seed composition) alone or together with food of the animal. In a preferred embodiment, the chia seed, chia seed composition comprises one or more of the following: dietary fiber, omega-3 fatty acid, vegetable protein, high calcium and iron content, high potassium antioxidant potential, and/or a substance capable of improving metabolism in type 2 diabetes.

According to another embodiment of the invention, the chia seed according to the invention (or an equivalent dose of chia seed composition) is administered orally in an amount of about 5 to about 100 grams per day, alone or mixed into the food administered before or during the meal. In another embodiment the chia seed (or equivalent dose of chia seed composition) is administered in an amount of about 10-100 g/day.

In yet another embodiment, the chia seed is administered, before, during or after a meal. Preferably it is administered at a time suitable to achieve the desired effect.

In yet anther embodiment, the chia seed is administered for a duration of time to achieve or maintain the desired effect. Such effect can be determined by monitoring the indicators of such an effect (i.e. blood pressure, blood glucose/insulin levels, t.PA, NOₓ levels (an an indicator of endothial function), fibmogin, factor VIII, (coagulation) von Willbrand factor, CRP, feritinin (iron status) other indicators listed in Tables 7 or 8).

According to yet another embodiment of the method of the invention the administration of chia seed, according to the invention is by a liquid, a powder, or as a part of a food product.

According to another aspect of the present invention the chia seed and chia seed compositions and methods of the invention can be applied to the treatment of long-term diabetes, atheroslerosis, heart disease, blood pressure, blood glucose, and anemia. In addition the compositions and methods of the invention provide methods for reduce inflammation, improve coagulation and fibrinolysis in an animal and of treating type 2 diabetes as well as for reducing systolic blood pressure. Such methods comprise the administration of an effective amount of chia sees to a patient or animal in need thereof.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will be better understood with reference to the drawings in which:

FIG. 1 illustrates nutrient equivalent of 10 g of Chia seeds with that of other foods.

FIGS. 2A and B are bar graphs illustrating the percent fatty acid profiles of chia seeds used in the studies (A) and flax seed (b) Analysis were performed at the University of Toronto, lipids research laboratories. PUFA is polyunsaturated fatty acid, MUFA is monounsaturated fatty acid; SFA is soluble fatty acids.

FIG. 3 is a linear graph illustrating the effects of the control (WB) and chia diet in Example 1 on post-meal blood glucose (plasma) response of individuals histogram

FIG. 4 is a linear graph illustrating the effects of the control (WB) and chia diet in Example 1 on post-meal blood insulin response of individuals histogram.

FIG. 5 indicates that the long term study utilized randomised, single blind, cross over designed, where approximately half of people were randomly assigned to received either control diet prescribed by Canadian Diabetes association and conventional medical treatment, and other half received the same diet in which Chia seed were incorporated to be consumed for 12 weeks. After 4 weeks of washout period the same patients were cross over to diet and followed for 12 weeks.

FIG. 6 is a bar graph illustrating the change of the primary parameter measured, glycolated haemoglobin A1C of Example 2.

### DETAILED DESCRIPTION OF PREFERRED EMBODIMENTS OF THE INVENTION

The details of the preferred embodiment of the present invention are set forth in the accompanying drawings and the description and examples below. Once the details of the invention are known, numerous additional innovations and changes will become obvious to one skilled in the art.

The present invention provides the results of controlled studies using chia seeds in human health, preferably Salvia Hispanic seeds especially in reduction of CVD (cardiovascular disease) risk factors such as diabetes, blood glucose levels, and blood pressure. Also, present invention provides the results of controlled studies on the effect of chia seeds and omega-3 fatty acids, especially of plant origin, such as chia seeds, on thromo-atheroslerotic factors such as inflammation, coagulation and fibrinolysis.

The present Inventor shows that chia seed has a significant application in the the control, management and treatment of certain medical considtions, such as those related to the factors in Tables 7 and 8 and especially those related to cardiovascular disease and diabetes. Chia seeds actually contain an oil rate varying between 27-33% and offers one of the highest percentage of α-linolenic acid (i.e. 60-70%) known in nature. It must be emphasized that α-linolenic acid is an unsaturated omega-3 fatty acid. These poly-unsaturated fatty acids like α-linolenic are very important as regards to human nutrition as they are not synthesized by the body and must be supplied in food. Foods including oils containing a high rate of omega-3 fatty acids can reduce the risk of cardiovascular disease.

Regarding other oleaginous crops, chia possesses the one of the highest percentages of poly-unsaturated fatty acids linolenic (i.e. 65-70%); this species is followed by flax with 49-54% of total oil content. Although canola also offers a high degree of unsaturation (67%), this issue arises from oleic (monosaturated) acid's high content thus showing a relatively low content (27%) of poly-unsaturated fatty acids.

Chia seeds comprise 21% (19-23%) of proteins. This percentage is favorably compared to other nutritional grains such as wheat (14%), corn (11%), rice (8.5%), oats (15.3%), barley (9.2%) and amaranth (6.7%). Unlike the above compared grains, chia's protein amino acids have no limiting features with regard to the adult diet, and contains all 9 essential amino acids in a most optimal proportion. In contrast, the above compared grains do have such limits as regards to two or more essential amino acids. Hence, the above compared grains must be mixed (cannot be used alone) to satisfactorily provide human amino acid needs.

Water and methanol extracts pertaining to degreased chia seeds have demonstrated a strong antioxidizing activity. Most important isolated antioxidants of this seed are chlorogenic acid, caffeic acid and flavonol glycosides.

The presence of anti-oxidants in chia seeds, as opposed to other seeds containing linolenic acid (i.e. flax seeds) that rapidly decompose due to the lack of anti-oxidants, results in a chia having a longer shelf life and a better food source.

After oil extraction, the remaining chia flour contains a 50-60% of fiber. Chia seed possesses 5% of soluble fiber which appears as mucilage when the seed is humidified.

Chia's chemical composition and/or nutritional value and medicinal value as shown byb the inventor herein, causes this species to possess applications within several food and industrial markets.

Although, there were previous anecdotal evidence linking North and South American indigenous diets, that include chia, in reducing the prevalence of diabetes in these native communities, expecially type 2 diabetes. The present inventor has determined scientifically that chia (Salvia Hispanica L.) seeds are able to reduce cluster of conventional and emerging risk factors assoiciated with diabetes and/or cardiovascular disease or other related conditions (other conditions in which such factors, as listed in tables 7 and/or 8 are indicative of). The present invention leads to new treatments and therapies for managing and reducing the risk of such conditions and to compositions that effect such treatments and therapies.

In summary, the present invention in certain embodiments provides a method for the treatment and/or management of diabetes and/or the treatment and management of cardio vascular disease or diabetes associated conditions or risk factors, such as one or more of the following: blood pressure, blood glucose levels, post-prandial glycemia, inflammatory factors (C-reactive protein), coagulation (fibrinogen, factor VIII), fibrinolytic factors such as t-PA, iron status and endothelial function or other conditions related to such indicators. In one embodiment the invention relates to dietary approaches to such treatment and management.

In a preferred embodiment, the methods of the invention comprise administration of an effective amount of chia seed, a chia seed composition or a chia seed-like composition to a patient in need thereof.

The term chia seed as used herein refers to any whole, ground or liquefied form of the chia (Saliva Hispanica L.) seed and includes chia seed compositions.

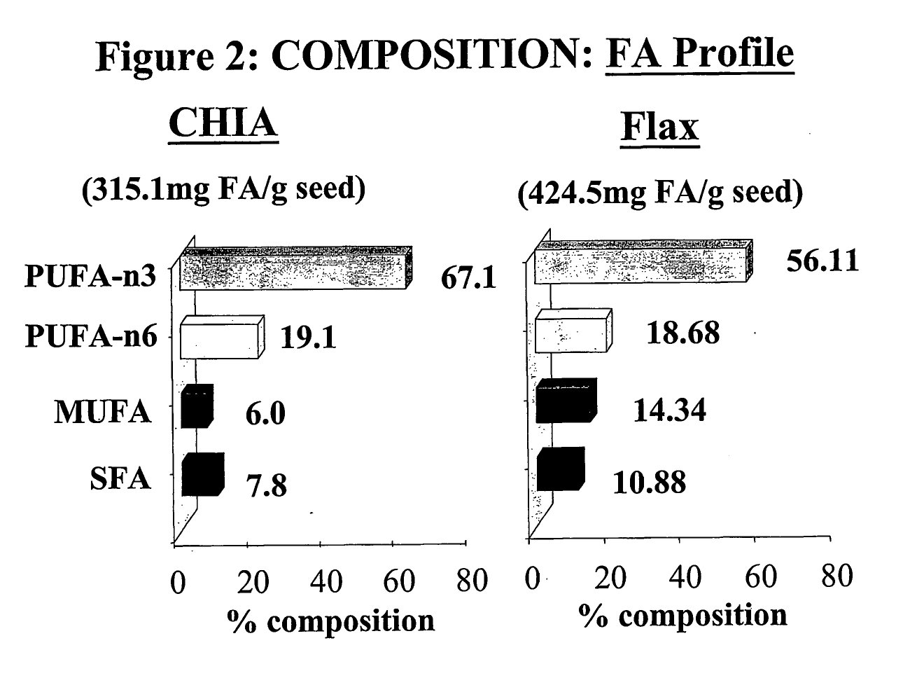

The term chia seed composition as used herein refers to a composition comprising chia seed (whole, ground, liquefied, or a desired active component(s) derived or extracted from chia seed). Such desired active components will depend on the factors to be controlled. In one embodiment, such compositions comprise the nutrient and/or fatty acid composition of Table 2 or FIG. 2. It can also include synthetic or chemical equivalents to such compositions that produce a similar effect. It can also include compositions in the form of food (i.e. breads, biscuits) and/or pharmaceutical type compositions.

A person skilled in the art would know how to make pharmceutical or pharmaceutical type compositions, suitable for the applications of the present invention. chia seed or chia seed compositions of the present invention may be administered in a convenient manner such as by oral administration (capsules, tablets, food, raw seed, ground seed, etc.). Depending on the route of administration, the active substance may be coated in a material to protect the compound from the action of enzymes, acids and other natural conditions which may inactivate the compound. If the active substance is a omega-3 fatty acid it may be delivered using techniques known in the art.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985) or Handbook of Pharmaceutical Additives (compiled by Michael and Irene Ash, Gower Publishing Limited, Aldershot, England (1995)). On this basis, the compositions include, albeit not exclusively, solutions of the substances in association with one or more pharmaceutically acceptable vehicles or diluents, and may be contained in buffered solutions with a suitable pH and/or be iso-osmotic with physiological fluids. In this regard, reference can be made to U.S. Pat. No. 5,843,456. As will also be appreciated by those skilled, administration of substances described herein may be by an inactive viral carrier.

Administration of a therapeutically effective, sufficient amount, or an effective amount of pharmaceutical compositions for chia seed, or chia seed composition of the present invention is defined as an amount effective, at dosages and for periods of time necessary to achieve the desired result. For example, a therapeutically effective, sufficient, or effective amount of a substance may vary according to factors such as the disease state, age, sex, and weight of the individual, and the ability of the substance to elicit a desired response in the individual. Dosage regimes may be adjusted to provide the optimum therapeutic response. For example, several divided doses may be administered daily or the dose may be proportionally reduced as indicated by the exigencies of the therapeutic situation. Preferred effective amounts of chia seed are 5-100 g/day, chia seed compositions that is equivalent to 5-100 g/day of chia seed. The regime could also include a mix or chia seed and chia seed compositions.

In another embodiment the amount administered is 10-100 g/day of chia seed or compositional equivalent thereto.

In another embodiment the chia seed and/or chia seed composition is administered in an effective amount and at an effective time, i.e. before, during or after a meal, as the case may be, in one embodiment before or during a meal is another embodiment 1-180 minutes before a meal, to obtain the desired results. A person skilled in the art would appreciate that in certain embodiments of the invention timing of administration of the chia seed, chia seed composition or chia seed like composition may in certain circumstances may be important to ensure that the desired active component(s) of said chia seed, chia seed composition or chia seed-like composition is present in the body at the critical time to have the desired effect. The timing of administration may also depend on the particular formulation of the chia seed, chia seed composition or chia seed-like composition. For instance, if chia seed or chia seed compositions are administered in the form of capsules, a person skilled in the art would appreciate that certain coatings or other factors may be used to effect the timing of the release of active components in the body.

As described above, in one embodiment, the present invention also relates to compositions and methods for reducing blood glucose and blood pressure. In particular, the present inventor has found that chia seed is effective in the reduction of blood glucose, blood pressure, inflammation and coagulation factors.

In one embodiment, chia seed and/or chia seed composition(s) can play a metabolic role or affect one or more of the following: thrombosis, arrhythmia, inflammation, platelet aggregation, atherosclerosis and endothelium function.

In another embodiment the invention provides a use of omega-3 fatty acids, especially of plant origin, such as chia seeds or from chia seeds, on thrombo-atheroslerotic factors such as inflammation, coagulation and fibrinolysis. As such compositions or foods comprising omega 3-fatty acids or plants or seeds comrprising omega-3 fatty acids and methods for using an effective amount of the same are included within the scope of the present invention.

More particularly, the chia seed and/or chia seed compositions can be used to:

(i) control or manage blood glucose levels, preferably postprandial glucose levels, preferably reduction of blood glucose levels. Preferably the chia seed, chia seed compositions or chia seed-like composition is administered before or during a meal.

(ii) control or mange fibrinogen, factor VIII and/or vWLBR factor, preferably reducing levels of such factors, preferably blood levels of such factors.

(iii) control or mange t-PA and/or PAI-I levels, preferably increasing such levels, preferably blood levels.

(iv) control or manage CRP levels, preferably reducing such levels, preferably blood levels.

(v) control or manage feritinin levels, preferably increasing such levels, preferably blood levels.

(vi) control or manage fasting glucose levels, preferably reducing such levels, preferably blood levels.

(vii) control or manage nitric oxidelevel, preferably reducing such levels.

(viii) control or manage systolic blood pressure levels, preferably reducing such levels.

(ix) control or manage diastolic blood pressure levels, preferably reducing such levels.

(x) control or manage or treat or reduce risk of development, of any conditions associated with any one or more of the above-noted indicators listed in (i)-(ix), such as glycemia, diabetes, cardiovascular disease, inflammation, fibrinolysis, coagulation, endothelial function, thrombosis, arrhythmia, platelet aggregation, atherosclerosis, or iron status.

In one embodiment, chia seed and/or chia seed compositions can be used to control said factors in both non-diabetic and diabetic individuals. Such uses and methods are intended to be included within the scope of the present invention.

In one embodiment said chia seeds or compositions comprise the nutrient and/or fatty acid profile of Tables 2 or FIG. 2. In another embodiment, said seeds or compositions comprise the active component necessary to affect the desired effect, preferably in the proportion noted in said Tables. For instance to increase iron levels, the desired iron content should be maintained along with potentially other factors that may affect absorption of iron in the body

As used herein “patient” and “animal” means any member of the animal kingdom including preferably humans, that would benefit from the use of the chia seed, chia seed compositions or chia seed-like compositions of the invention, or the methods of the present invention.

As used herein “postprandial” means after any food intake.

As used herein “sufficient amount” means an amount of a composition, substance or reactant to give an observable result, including desired results

As used herein “during or before a meal” means at any time after the commencement of consumption of one or more pieces of food by an animal, and can be coincident with commencement, and before the end of consumption of all food consumed by the animal, at one sitting or occasion and can be coincident with completion of consumption or immediately thereafter.

As used herein “a food” means any substance or composition of substances or compounds which are usually consumed by an animal, preferably for some nutritional value.

As used herein “a meal” means the consumption of one or more morsels or pieces of a food in a sitting where a sitting is the time taken to consume the one or more morsels or pieces of a food.

As used herein “consumed alone” or “together with food” means that Chia seeds, chias seed compositions or chia seed-like compositions could be taken in either way to be effective.

In another embodiment of the invention, the invention provides a treatment regime for controlling, managing, treating or reducing risk of any the aforementioned conditions comprising adminstration of chic seed and/or for chic seed compositions at an amount of about 5-100 g/day, for instance it can be incorporated into food, sprinkled on food, eaten or consumed alone, before, during or after a meal.

The following non-limiting examples are illustrative of the present invention:

### EXAMPLES

### Chia Seeds Used in Examples 1-3

Chia seeds used in the following examples were grown in South America and received from the Chianova Company from Toronto, Ontario, Canada.

A complete energy content and nutrient composition analysis of the chia seeds used in the examples was conducted by the University of Guelph. The results of the analysis is shown in Table 2. It is believed that the potential physiologically active components in Chia include soluble and dietary fiber, omega-3 fatty acids, high level of protein, high potassium content, calcium, and iron, but also high potency antioxidants, and flavonoids.

According to the University of Guleph laboratory analysis Chia seed used on the short and long term study described herein contained 4.7 mg of ascorbic acid per gram of seed (an anti-oxidant).

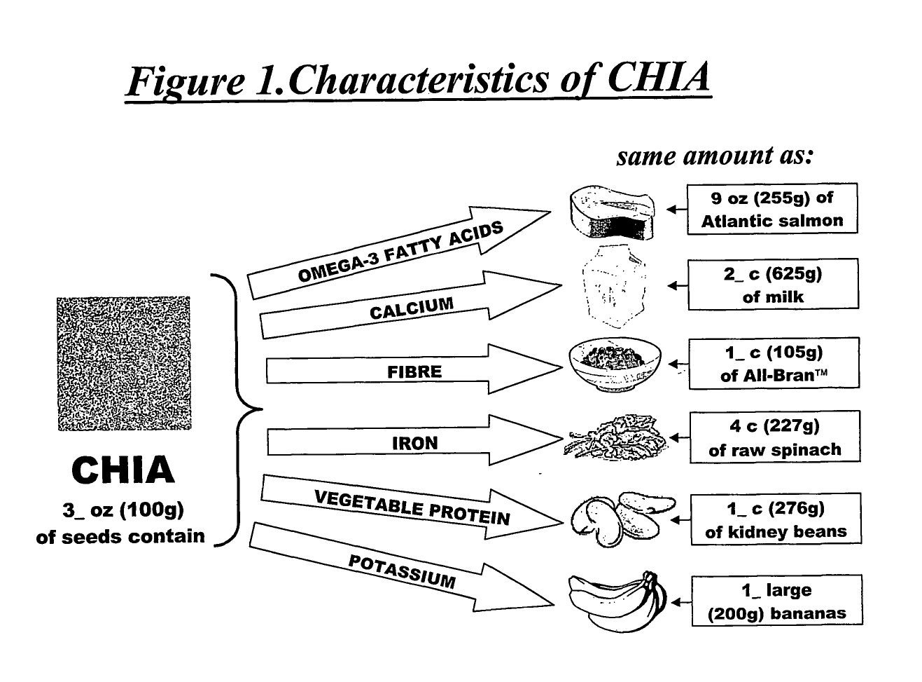

FIG. 1 shows the nutritional equivalent of 100 g of Chia as compared to other foods.

FIG. 2 illustrates the fatty acid composition of chia seeds (A) and flax seeds (1). It was proposed that the chia seed has a similar composition to flaxseed (Linum usitatisimsum) and thus may have similar effects on carbohydrate metabolism.

In the following examples (depending on the example), chia seed was administered in the following form: as a ground powder, alone as whole seed or ground powder, consumed alone, sprinkled on a meal, incorporated as supplement in bread or other foods regularly consumed by people. Some subjects in the example 2 study developed there own recepies by including Chia in omelets/eggs, muffins, cookies, or other foods, the criteria being to ensure a dosage regimen of about 5-100 g/day of chia seed was maintained, (no matter what the form). On average consumption in the long term study was about 50 g/day. In Example 1, the subjects were provided with the requisite amount of chia bread or control as the case may be.

When measured in complete seeds, total dietary fiber content of the chia diet in the following examples was 36, of which 2.3 g derived from soluble fiber (see Table 2). Although there is only 2.3 g of soluble fiber in seeds, the gel-forming capacity per gram of Chia seed soluble fiber is exceptional. Compared to viscosity of other soluble fiber, 1 g of soluble fiber are 11 times of Psyllium, 6 times of guar, and 2 times stronger then purified glucomannan. The importance of this nutritional seed is focused not only on its nutritional value but also on its “thickening nature” within the cosmetologic industry and within other applications. From the composition of seeds used in the study as shown in Table 2, it is interesting to note high content of potassium, calcium and iron.

### Chia Diet Used in Example 1

Example 1 was a one meal experiment. Chia seed incorporated into white bread containing 50 grams of available carbohydrate from white bread. And other test (chia) was the same except 20 grams of chia seeds was added to the same portion of white bread as used on control meal.

### Chia Diet Used in Example 2

In example 2 two different diets as shown in table 6, the test diet containing approximately on average 50 g chia per day (5-100) g/day and the control diet that was a conventional diet recommended by the Canadian Diabetes Association. Part of the calories from the Canadian Diabetes Association diet were replaced by chia in the test phase of the study. The difference between the 2 diets are shown in table 6.

### Example 1

### Postprandial Effect of Chia (Acute Clinical Study)

Subjects and Methods

Twelve healthy fasting males (age: 39.5±4.5 years, BMI: 25.8±0.9 kg/m²) consumed either a standardized dose of 50 grams of white bread (WB) containing 50 g of available carbohydrate or the same prepared with 20 g of whole Chia in a randomized-crossover-design. The composition is irrelevant because it is identical with difference of 20 grams of chia to standard bread. Example 1 was conducted after fasting. Chia is added to bread and baked together. Chia is 20 grams per serving. Protocol is the same means fasting blood and the measurements taken at 15, 30, 60, 90 and 120 minutes after consuming chia or control bread.

Fatty acid (FA) composition of Chia (Table 2, also see FIG. 2) was determined by the University of Guelph, Ontario, Canada. Chia was provided by Agropecuaria El Valle S. A, Argentina. Total FAs were extracted. FA methyl esters were then prepared and measured using gas chromatography. The clinical testing protocol followed established glycemic index testing guidelines. [Wolever Tms, Jenkins Dja, Jenkins Al, Josse Rg. The Glycemic Index Methodology, Am J Clin Nutr 1991;54:846:54.]

Results and Discussion

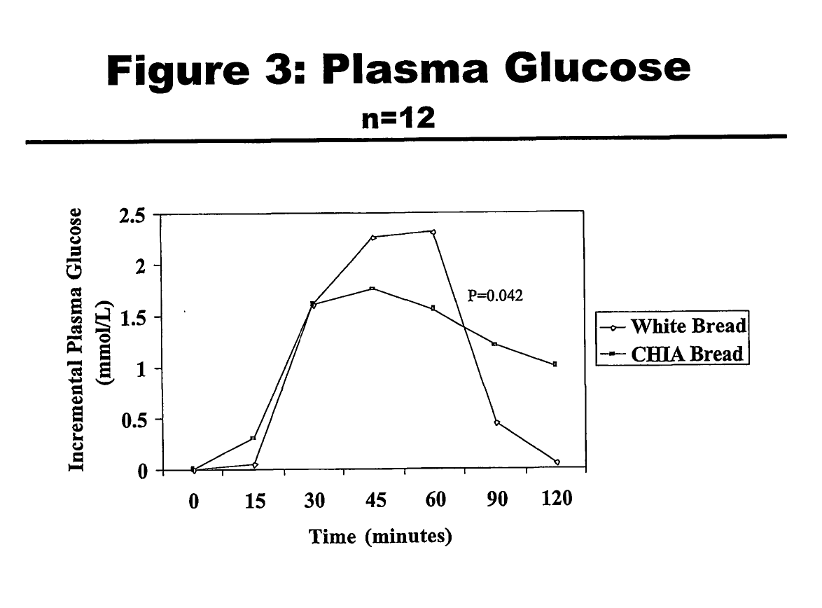

Glycemic testing demonstrated that bread supplemented with Chia Seed (CS) increased incremental glycemia at 90 min compared with WB (1.3±0.3 vs 0.4±0.4 mmol/L, p=0.04). Conversely, it lowered incremental insulinemia at 30 min (24.7±8.3 vs 47.5±14.4 pmol/L, p=0.57) and 45 min (72±14.9 vs 119±20.0 pmol/L p=0.02) compared with WB. There was no effect of Chia on the area under the curve for glycemia (145.5±22.2 vs 133.3±30.0 mmol/L) or insulinemia (5909±922 vs 6677±1148 pmol/L). FIG. 3 is a graph illustrating post-meal blood gluose effects of control (white bread consumption—WB) and Chia bread consumption. Blood samples were taken at every baseline and then at 15-30 minutes as in figures.

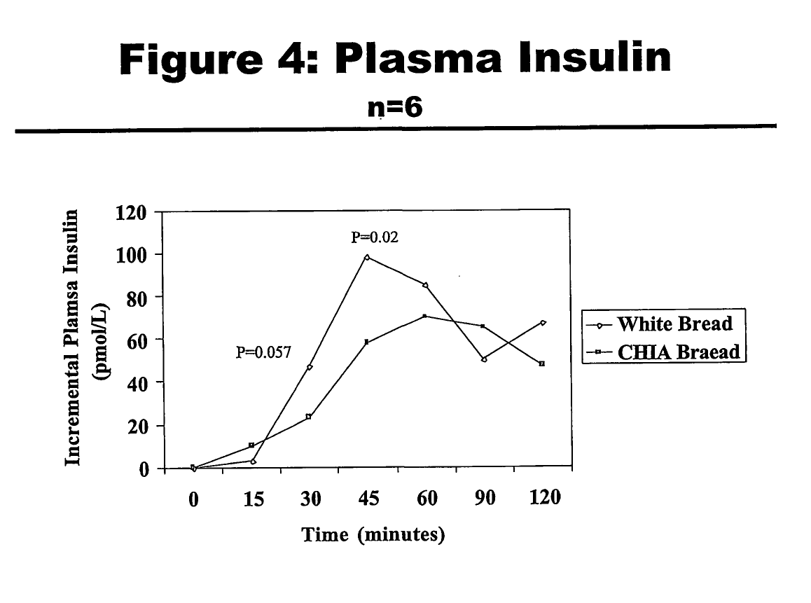

FIG. 4 is a graph illustrating the post-meal blood insulin effects of control (WB) and chia bread consumption.

These results indicated a reduction in postprandial glucose and insulin levels and is indicative of insulin insensitivity.

Chia is a rich plant source of α-linolenic acid and other important nutrients. Together the higher glycemic profile in the last 30 min and lower insulinemic profile in the first 45 min following Chia suggest that it might prolong glucose absorption in the gut. This preliminary data supports further interest to study chia in a long term study in individuals with type 2 diabetes.

### Example 2

### Long Term Study Pertaining to the Efficacy and Safety of Chia Seed in Type 2 Diabetes

1.0—Summary

The following study was conducted to determine the effect of the addition of Salvia Hispanica (Chia) seeds to the Canadian Diabetes Association (CDA) diet (which recommends to consume 55% of calories from carbohydrate, 15 from protein and 30% from fat) and conventional medical treatment associated with improvements in diabetes control, as assessed by HbAlc, blood glucose and plasma insulin concentrations, and to determine the effects on blood pressure, plasma lipids, especially inflammation, fibmolysis, coagulation factors, and quality of life. Twelve-week metabolic studies were used to assess the effect of chia seeds on glycemic control, blood pressure and serum lipids in subjects with type 2 diabetes. Addition of chia seeds to regular treatment was associated with a lowering in 24 h urinary C-peptide excretion (as a marker of insulin secretion) and improvement in inflammation, fibrinolysis, coagulation factors, and quality of life

2.0 Subjects and Methods

2.1 Subjects Recruitment

Otherwise healthy type 2 diabetic men and postmenopausal women (to reduce effect of hormones and complication regarding patients scheduling) were recruited by newspaper advertisement, physician referral and the diabetic clinic at St. Michael's Hospital.

2.2 Inclusion Criteria

Inclusion Criteria are summarized in Table 3. HbAlc between 6.5 and 9% at recruitment (i.e. below 140% of the upper limit of normal which is recognized as the upper limit of acceptable control), living within a 40 km radius of the test center (St. Michael's Hospital) and on diet alone or diet and glyburide/glipizide. Previous studies have shown that α-glucosidase inhibitors such as Acarbose have a comparable effect on HbAlc in diabetic subjects on diet alone or diet plus oral agents. That level of reduction is clinically significant due to its beneficial effect on reduction of diabetes related complications.

2.3 Exclusion Criteria

Exclusion Criteria are summarized in Table 3. Diabetic complications: clinically significant gastroparesis, retinopathy, nephropathy, neuropathy, hepatic disease or CHD; taking insulin or hormone replacement therapy, BMI>38 kg/m², smoking or significant alcohol intake (>2 drink/day), serum triglycerides≧4.0 mmol/L or using α-glucosidase inhibitors. Previous studies have shown that α-glucosidase inhibitors have the same effect on HbAlc in diabetic subjects on diet alone or diet plus oral agents. Individuals that change their regular anti-hypertensive and cholesterol-lowering medication are excluded from the study.

2.4 Power (Subjects n=28)

Assuming a 30% attrition rate, to detect a treatment difference of 0.75% in HbAlc, 28 men and women were used for the study (assuming alpha=0.05 and beta=0.8, n=29 subjects). The assumptions behind the calculation were the following: a) high carbohydrate diet (control) will have no effect on HbAlc levels. b) high Chia supplement containing 50 g of finely ground Chia may reduce HbAlc by 0.50% which is a result similar to a published study of the effect of acarbose on HbAlc levels as a model of a modest food-like effect The standard deviation of 1.23 for percent change in HbAlc has been used in sample size calculation, in line with previously published results.

2.5 Initial Treatment

Those subjects that were deemed to be potentially eligible for the study were asked to give a fasting blood sample at the Risk Factor Modification Center after completing a 1-week diet history. Individuals, who met the study criteria, were invited to return again to the Center. The principles of the diabetic diet which they are already expected to be following will be reinforced, which incorporate the key elements of an NCEP Step 2 diet (total calories from fat <30%, saturated fat <7%, polyunsaturated fat <10%, dietary cholesterol <300 mg/day). The NCEP Step 2 diet is recommended by the American Heart Association. Potential subjects whose HbAlc levels remained within the inclusion range in the 1-2 months prior to the metabolic diets were retained and provided with self tarring digital scales in order to obtain weighed diet histories during the first week prior to starting the study and to use while recording subsequent diet histories. The demographic profile of the subjects involved in the study can be found at Table 4.

3.0 Protocol

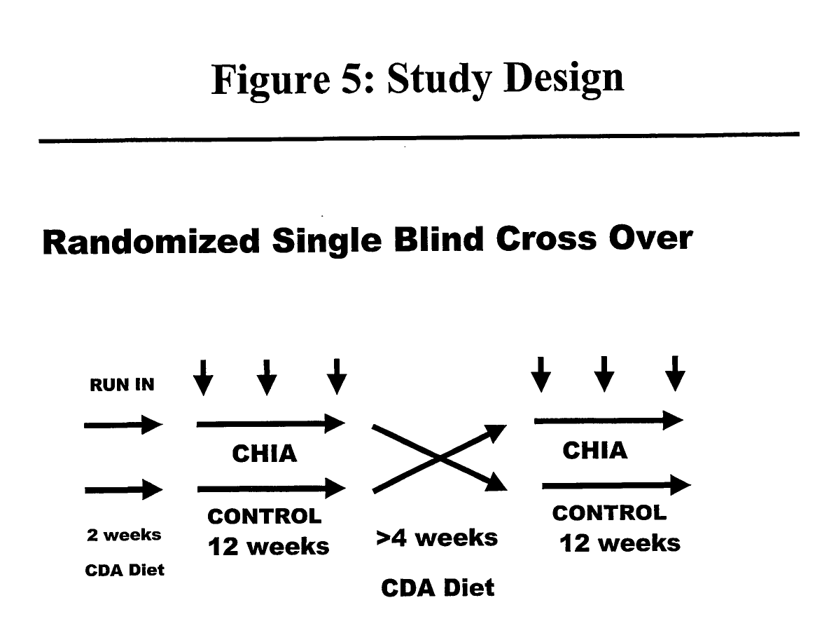

All subjects underwent two 12-week a single-blind treatments in random order (using computer generated randomization tables) in crossover design. [SEE FIG. 5]. In addition to subject selection and exclusion criteria, variables that were controlled during the study are summarized in Table 5.

1.1 Treatments:

1) CDA high carbohydrate diet (approximately 55:15:30% of CHO:Protein:Fat of energy intake). To match the fiber content on control, equivalent content of fiber were added from AACC certified Hard Red Spring Wheat Bran.

2) high Chia supplements (containing 25 g/1000 kcal of Chia seeds with plateau of 100 g/day). (i.e. Chia was administered based on nutrient/energy basis, or according to the participants food consumption. They received 25 g of chia per each 1000 k cal of food they consume. Those who consumed more then 4000 cal per day did not receive more then 100 g of chia but only 100 g maximum per day).

3.2 Duration

The study consisted of two months recruitment and patient selection, estimation of individual caloric requirements; two 12-week treatment periods separated by a washout of at least one month duration. Total duration: 8 months per subject.

3.3 Study Details

Fasting blood samples were obtained at day zero and weeks 2, 4, 6, 8, 10 and 12 of each study period. Twenty-four hour urine for urinary C-peptide analyses, 24 hr blood pressure monitoring, and quality of life questionnaire were obtained immediately prior to the beginning of the study and at the end of each 12-week treatment phase.

4.0 DIETS

Diets were the subjects' diabetic diets, which conformed to CDA and NCEP Step 2 guidelines. Diet histories were recorded at weeks 2, 4, 6 and 8. The dietitian assessed these diets for consistency in the subject's presence. The week-2 diet plan of the first phase was photocopied, returned to the subject and used to establish the eating pattern of the subject for the rest of the study. Where necessary, modifications in diet were made to ensure weight maintenance.

4.1 Supplements

These consisted of wheat bran and Chia seed enriched breads together with muffins developed by ChiaNova Research Corp. Both, wheat bran and Chia seed are safe for human consumption because of long history of its consumption in America. Possible gastrointestinal side effects may develop, including an increase in bowel movement, and in rare cases, mild diarrhea. Approximately 30% of total test or control supplements were given to study participants to be mixed with their regular foods, such as mashed potatoes, yogurt etc. (e.g. one supplement is whole or ground chia, other in control phase of diet is wheat bran, skim milk powder to match for protein and fiber content of chia supplement) The test supplements deliver 25 g of chia per every 1000 kcal diet. The control supplements (AACC standardized Red Spring wheat bran) matched the test supplements for total dietary fiber. The test supplements deliver approximately 12 g of unsaturated fat, and 2 g of dietary fiber per 1000 kcal dietary energy daily, while the control supplement provided 2 g of dietary fiber per 1000 kcal. This difference between test and control is more than 15% times the increase in unsaturated fat intake which was shown in the Nurses and Health Professionals Studies (Walter Willett et al.) to be associated with a reduction to half the relative risk of developing heart disease and stroke over a 6-year period. Supplements developed, tested for palatability and analyzed for macronutrients prior to the commencement of the study. The nutrient profile of actual intake between control and chia enriched interventional diet is summarized in Table 6.

4.2 Compliance

Compliance was assessed by records of supplements consumed and from the return of any food items not consumed.

5.0 Outcomes

A list of the parameters of interest is summarized in table 7.

5.1 Primary: markers of glycemic control: HbAlc, fasting plasma glucose.

5.2 Secondary: fasting blood glucose, insulin, 24 hr. urinary glucose excretion, blood pressure, serum triglyceride, LDL-C, HDL-C, apo B, apo AI. Also, other markesr measured include nitric oxide (endothelial function), high-sensitivity C-reactive protein (inflammation), fibrinogen, factor VIII, and vonWillenbrand factor (coagulation), and fibrinolytic factors (TPA and PAI-I).

5.3 Safety: The main safety parameters included liver function (AST, ALT), kidney parameters (urea, creatinine) and bleeding time (all major parameters).

6.0 Measurements

6.1 Blood

12 h fasting blood samples were obtained prior to the start and on weeks 2, 4, 6, 8, 10 and 12 of each metabolic phase for plasma glucose, HbAlc and insulin. Samples were analyzed for serum FFA, insulin and C-peptide (wk 0, 12). Plasma lipids and lipoproteins were measured following ultracentrifugation; serum apo AI and B, and amino acids (wk 0 and 12) were measured on frozen serum stored at −70° C. Other analysis performed included nitric oxide (endothelial function), high-sensitivity C-reactive protein (inflammation), fibrinogen, factor VIII, and vonWillenbrand factor (coagulation), and fibrinolytic factors (TPA and PAI-I).

6.1 Urine

24 h urine collections were obtained immediately prior to and at weeks and 12 of each metabolic phase for measurement of creatinine, urea and C-peptide outputs.

6.2 Diet History

One-week weighed diet histories were obtained prior to the start of each metabolic phase and assessed for macronutrients, dietary fiber and fatty acids. Completed bi-weekly

6.3 Anthropometric

Height at recruitment and waist and hip circumference, and body composition were taken immediately prior to and at the end of each study phase. Body weight and blood pressure were measured at bi-weekly intervals throughout.

6.4 Quality of Life

Validated questionnaire for the quality of life of type 2 diabetic patients were assessed at the beginning and end of each treatment periods.

7.0 Quality Control

Control and supplements were analyzed for macronutrients, fiber and fatty acids content.

8.0 Statistical Analysis

The results are seen in Table 8 and are expressed as mean±standard error. The treatment effect was assessed by analysis of variance/covariance facility within the general linear model package—PROC GLM/SAS (SAS/STAT Users' Guide, vol. 2, 1998). The model specification, appropriate to split-plot analysis, posits the end-of-treatment measurement as response variable, treatment, sex and treatment sequence as main effects, random term due to subject nested within sex by sequence interaction and where applicable, a covariate term due to baseline value. Furthermore, the degree of linear association between responses of various risk factors and levels of macronutrients as well as anthropometric data were tested through Pearson as well as partial correlation (PROC CORR/SAS). Additionally, paired Student t-tests were performed to assess changes across treatment for response variables that will comprise the descriptive statistics tables.

9.0 Results and Discussion

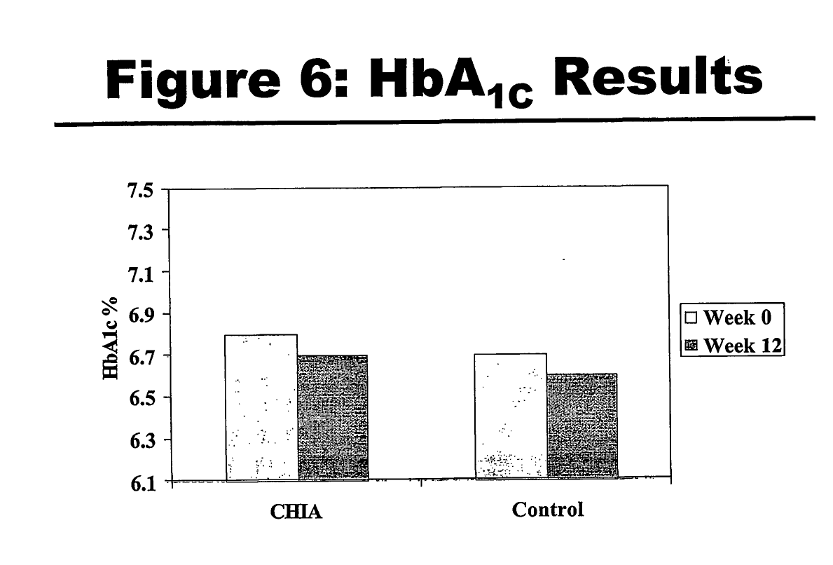

The results of the study are summarized in Table 8. Table 8 provides all parameters measured, presented at start and end of each study period with level of significance presented across each interventional period (symbol * means significant), as well as P values expressed as difference between control diet and Chia diet intervention periods. HbAlc levels are illustrated in FIG. 6.

Based on limited feeding studies showing improvements in carbohydrate tolerance and the findings from large cohort studies that high plant sources of omega-3, high unsaturated fat, and fiber intakes (Harvard study, Garg and S. Grundy) protect from the development of type 2 diabetes and heart disease. In preliminary study the group assessed the effect of 30% Chia enriched bread on postprandial glycemia in ten healthy volunteers [Examples 1]. The Chia bread significantly lowered area under curve for glucose and reduced insulin response at time 30′ and 45′ compared to control bread (FIGS. 3 and 4) (Bazinet R P, Sievenpiper J L, Stavro M P, Cunnane S C, Vuksan V. Chia (Salvia Hispanica L.) seeds rich source of α-linolenic acid prolongs posprandial glycemia. FASEB J 15(758.1): A992, 2001). Based on these preliminary results a long term study was conducted (Example 2).

In the long term study, the metabolic parameters of interest included measurements of glycemic control (HbAlC, plasma glucose), marker endothelial function (nitric oxide), inflammation (high-sensitivity C-reactive protein), coagulation (fibrinogen, factor VIII, and vonWillenbrand factor), and fibrinolysis (TPA and PAI-I). The results showed that diets high in Chia will result in improved carbohydrate tolerance indicated by reductions in serum HbAlc, with benefits on blood pressure, blood glucose levels, post-prandial glycemia, and endothelial function, inflammation, coagulation and fibrinolysis. It also showed improved iron status (levels).

The present inventor has found that chia has long-term overall metabolic effect that are beneficial in a number of ways. Chia has a favorable nutrient composition that include high level of omega-3 fatty acids, vegetable protein and dietary fiber, high viscous fiber, calcium, and potassium. The results support advice to diabetics and those at risk of diabetes (family history, overweight, impaired glucose tolerance) or related conditions, such as cardiovascular disease or other conditions related to levels of various parameters measured herein and listed in Table 8 [e.g. glycemic control (HbAlC, plasma glucose), marker endothelial function (nitric oxide), inflammation (high-sensitivity C-reactive protein), coagulation (fibrinogen, factor VIII, and vonWillenbrand (vWLBR) factor), fibrinolysis (TPA and PAI-I).] and iron status to increase their consumption of high unsaturated fat/high omega-3 products, rich in vegetable protein and dietary fiber.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Summary of Dietary Intervention Studies
Results of sucesseful inteventional studies in which varing content of
omega-3 was given with respect to percent of heart disease mortality
reduction (In our study the participants consumed 50 g of Chia per day
that provided an equivalent of about 9.4 g of omega-3)

| STUDY | N-3 INTAKE | Δ MORTALITY |
|---|---|---|
| Lyon heart Study | 0.81 g/d | 65% |
| NHLB1 | 1.14 g/d | 56% |
| Margarin | 6.3 g/d | NS |
| Nurses' Health Study | 1.36 g/d** | 45% |

Canola Margarine
**70% f total n-3 from ALA

TABLE 2
Energy Content and Nutrient Composition of 100 g of Chia
(1 Serving Size) Used In The Examples.
(Conducted By the University of Guelph)

| Amount Per Serving | % Daily Value |
|---|---|
| Calories | 500 |
| Fat 28 g | 43 |
| Saturated Fat 2.7 g | |
| PUFA 23 g | |
| n-3 17 g | |
| n-6 5.9 g | |
| MUFA 2.3 g | |
| Cholesterol 1 mg | 0 |
| Sodium 200 mg | 13 |
| Potassium 694 mg | 6 |
| Carbohydrate 40 g | 13 |
| Fibre 36 g | 144 |
| Soluble Fibre 2.3 g | |
| Insoluble Fibre 33.6 g | |
| Protein 21 g | |
| Vitamin A | 0 |
| Vitamin C | 6 |
| Ca | 70 |
| Fe | 50 |

TABLE 3
Diabetic Subject Selection and Exclusion Criteria For Long Term
Chia Study

Subject Selection
Individuals with Type 2 Diabetes
HbAlc = 6.5-9.0%
Diabetes controlled by diet alone or OHA (Oral
Hypoglycemic Agents)
Received ethics approval from SMH
Subject Exclusion Criteria
Taking exogenous insulin
BMI > 38 kg/m²
Using alpha glucosidase inhibitors
Smoker
Hormone replacement therapy
Micro-vascular complications or recent MI/stroke
Taking flax seed or fish oil

TABLE 4
Summary of the demographic and medical characteristics of subjects
that completed the long term study. N = 21

| CHARACTERISTICS | MEAN +/− SD |
|---|---|
| Age | 64 +/− 8 years |
| Males | 12 |
| Females | 9 |
| BMI | 28 +/− 4 kg/m² |
| HbA1C | 6.8 +/− 0.9% |
| Aspirin Use | 6 |
| BP Meds | 11 |
| OHA Use | 16 |

Parameters controlled and kept unchanged during entire course of
10 months of the long term Chia study.

CONTROLLED VARIABLES
Weight
Body Composition
Exercise
Diet
Prescription and OTC (over the counter) Medications

TABLE 6
Nutrient profiles of actual intake between control and Chia enriched
interventional diet

| CHIA | CONTROL |
|---|---|
| 45% Carbohydrate | 58% Carbohydrate |
| 23% Protein | 19% Protein |
| 32% Fat | 27% Fat |
| 50 g salba | Approx. 1 g n-3 fatty acids |
| 10 g n-3 fatty acids | |

TABLE 7
Parameters of interest in long term Chia study.

PRIMARY
HbA1c
SECONDARY
Measures of glycemia
Lipids
Blood Pressure (BP)
Inflammatory Factors
Fibrinolytic Factors
TPA
PAI-1
Coagulation Factors
Fibrinogen
Factor VIII
Endothelial Factors
NOₓ
Endothelin-1

TABLE 8
Effect of Chia enriched diet compared with Control diet on parameters
of glycemic control, blood lipids, coagulation, fibrinolysis, inflammation
and safety parameters in 21 type 2 diabetic individuals

| | Chia | | | | |
|---|---|---|---|---|---|
| | Start | Chia | Control | Control | Chia vs. |
| Parameters | (wk-1) | End (wk12) | Start (wk1) | End (wk12) | Control. |
| 1. Fibrinogen | 3.51 ± 0.6 | 3.22 ± 0.6* | 3.28 ± 0.7 | 3.35 ± 0.7 | P < 0.041 |
| 2. Factor VIII | 1.03 ± 0.4 | 0.95 ± 0.3* | 0.85 ± 0.4 | 0.7 ± 0.4 | P < 0.023 |
| 3. vWLBR factor | 1.11 ± 0.3 | 1.02 ± 0.4 | 1.14 ± 0.6 | 1.26 ± 0.5 | P < 0.032 |
| 4. t-PA | 10.0 ± 0.5 | 10.4 ± 0.4* | 9.4 ± 0.4 | 8.9 ± 0.6 | P < 0.047 |
| 5. PAI-I | 17.1 ± 1.8 | 17.8 ± 1.4 | 16.4 ± 1.2 | 16.3 ± 1.6 | n.s. |
| 6. CRP | 2.92 ± 0.9 | 2.72 ± 1.5* | 2.61 ± 1.9 | 3.29 ± 0.9 | P < 0.02 |
| 7. T-Cholesterol | 4.96 ± 1.1 | 4.87 ± 1.2 | 4.92 ± 1.3 | 4.94 ± 1.1 | n.s. |
| 8. HDL-C | 1.27 ± 0.2 | 1.20 ± 0.1 | 1.22 ± 0.2 | 1.21 ± 0.2 | n.s. |
| 9. Triglyceride | 1.68 ± 0.8 | 1.64 ± 1.1 | 1.77 ± 0.9 | 1.73 ± 0.8 | n.s. |
| 10. Apo - A | 1.65 ± 0.2 | 1.57 ± 0.2 | 1.57 ± 0.2 | 1.55 ± 0.2 | n.s. |
| 11. Apo - B | 0.99 ± 0.4 | 0.9 ± 0.5 | 0.7 ± 0.4 | 1.01 ± 0.2 | n.s. |
| 12. AST | 25.1 ± 11 | 24.5 ± 12 | 23.2 ± 12 | 23.0 ± 11 | n.s. |
| 13. ALT | 28.3 ± 12 | 28.1 ± 11 | 26.3 ± 10 | 26.1 ± 12 | n.s. |
| 14. Urea | 5.67 ± 1.3 | 5.55 ± 1.4 | 6.10 ± 1.0 | 5.74 ± 1.8 | n.s. |
| 15. Creatinin | 80.1 ± 32 | 78.8 ± 39 | 76.4 ± 39 | 78 ± 32 | n.s. |
| 16. Feritinin | 116 ± 66 | 114.8 ± 86* | 132 ± 122 | 104.8 ± 96 | P < 0.034 |
| 17. Fasting glucose | 7.73 ± 1.4 | 7.39 ± 1.7 | 7.68 ± 2.3 | 7.92 ± 1.8 | P < 0.048 |
| 18. Fasting Insulin | 83.2 ± 36 | 84 ± 44 | 75.2 ± 39 | 86.5 ± 32 | n.s. |
| 19. Nitric Oxide | 73 ± 26 | 62 ± 26* | 73 ± 26 | 73 ± 26 | P < 0.034 |
| 20. Systolic BP | 138 | 127* | 131 | 134 | P < 0.001 |
| 21. Diastolic BP | 85 | 81* | 78 | 80 | P < 0.042 |
| 22. HbA1C | 6.78 ± 1.8 | 6.73 ± 1.2 | 6.73 ± 1.5 | 6.61 ± 1.4 | n.s. |

*means p < 0.05

### CITATIONS FOR REFERENCES REFERRED TO IN THE SPECIFICATION

Alison K, Ryttig K R, Hylander B, Rossner S: A dietary fibre supplement in the treatment of mild hypertension. A randomized, double-blind, placebo controlled trial. J Hypertens 10:195-199, 1992

American Diabetes Association (ADA): Nutrition Recommendations and principles for people with diabetes mellitus. Diabetes care 22:S42-S43, 1999

Anderson J W, Tietyen-Clark J: Dietary fiber: hyperlipidemia, hypertension, and coronary heart disease. Am J Gastroenterol 81:907-919, 1986

Aro A, Uusitupa M, Voutilainen E, Hersio K, Korhonen T, Siitonen O: Improved diabetic control and hypocholesterolaemic effect induced by long-term dietary supplementation with guar gum in type 2 (insulin-independent) diabetes. Diabetologia 21:29-33, 1981

Brown L, Rosner B, Willett W W, Sacks F M: Cholesterol-lowering effects of dietary fiber: a meta-analysis. Am J Clin Nutr 69:3042, 1999

Burt V L, Cutler J A, Higgins M, Horan M J, LaBarthe D, Whelton P, Brown C, Rocella E J: Trends in the prevalence, awarness, tretment and control of hypertension in the adult US population: data from the Health Examination Surveys, 1960-1991. Hypertension 26:60-69, 1995

Cunnane S C, Hamdeh M J, Liede A C, Thompson L U, Wolever T M S, Jenkins D J A: Effect of Flaxseed on Lipid Metaboluism in hyperlipidemic individuals. Am J Clin Nutr. 61;62-68, 1995

DCCT Research Group: The effect of intensive treatment of diabetes on the development and progression of long-term complications in insulin-dependent diabetes mellitus. The diabetes control and complications trial. New Engl J Med 329:977-986, 1993

Eastwood M A, Morris E R. Physical properties of dietary fiber that influence physiological function: a model for polymers along gastrointestinal tract. Am J Clin Nutr 55:436-442, 1992

Ebihara K, Schneeman B O: Interaction of bile acids, phospholipids, cholesterol and triglycerides with dietary fibers in the small intestine of rats. J Nutr 119:1100-1106, 1989

Epstein F H: Plasminogen-Activator Inhibitor Type 1 and Coronary Artery Disease. NEJM 342;1792:1801, 2000

Friedewald W T, Levy R I, Fridrickson D S: Estimation of plasma low-density lipoproteins, cholesterol concentration without use of the preparative ultracentrifuge. Clin Chem 18:499-502, 1972.

Fruchart J C, Kora I, Cachera C, Clavey V, Duthilleul P, Moschetto Y: Simultaneous measurements of plasma apolipoproteins A-1 and B by electroimmunoassay. Clin Chem 28:59-62, 1982.

GISSI_Prevenzione Investigators. Dietery supplementation with n-3 PUFA and vitamine E after myocardial infarction: results of the GISSI_Prevenzione 354;447:455,1999

Goldsmith M G, Barrett-Connor E, Edelstein S L, Wingard D L, Cobin B T, Herrman W H: Dislipidemia and ischemic heart disease mortality among men and women with diabetes. Circulation 89:991-997,1994

Gu K, Cowie C C, Harris M I: Mortality in Adults With and Without Diabetes in a National Cohort of the U.S. Population, 1971-1993. Diabetes Care 21:1138-1145, 1998

Haffner S M, Stern M P, Hazuda H P, Rosenthal M, Knapp J A, Malina R M: Role of obesity and fat distribution in non-insulin-dependent diabetes mellitus in Mexican Americans and non-Hispanic whites. Diabetes Care 9:153-161,1986.

Haffner S M, Stern M P, Hazuda H P, Mitchell B D, Patterson J K: Cardiovascular risk factors in confirmed prediabetic individuals. Does the clock for coronary heart disease start ticking before the onset of clinical diabetes? JAM4 263:2893-8, 1990

Haffner S M, Lehto S, Ronnemaa T, Pyorala K, Laakso M: Mortality from coronary heart disease in subjects with type 2 diabetes and in nondiabetic subjects with and without prior myocardial infarction. N Engl J Med 339:229-34, 1998

Harris M I, Flegal C M, Cowie C C, Eberhardt M S, Goldstein D E, Little R R, Weidmeyer H-M, Byrd-Holt D D: Prevalence of diabetes, impaired fasting glucose, and impaired glucose tolerance in U.S. adults: The Third National Health and Nutrition Survey, 1988-1994. Diabetes Care 21(4):518-524, 1998

Helmrich S P, Ragland D R, Leung R W, Paffenbarger R S Jr.: Physical activity and reduced occurrence of non-insulin-dependent diabetes mellitus. N Engl J Med 325:147-152, 1991

Himswarth H: Diabetes mellitus: a differentiation into insulin-sensitive and insulin-insensitive types. Lancet i: 127-130, 1936

Hochberg Y: A sharper Bonferroni procedure for multiple test significance. Biometrika 75:800-802, 1988

Hunninghake D B, Stein E A, Dujovne C A, Harris W S, Feldman E B, Miller V T, Tobert J A, Laskarzewski P M, Quiter E, Held J, Taylor A M, Hoffer S, Leonard S B, Brewer B K: The efficacy of intensive dietary therapy alone or combined with lovastatin in outpatient with hypercholesterolemia. N Engl J Med 328:1213-1219, 1993

Jenkins D J, Wolever T M, Leeds A R, Gassull M A, Haisman P, Dilawari J, Goff D V, Metz G L, Alberti K G: Dietary fibres, fibre analogues, and glucose tolerance: importance of viscosity. Br Med J 1:1392-4, 1978

Jenkins D J A, Wolever T M S, Rao A V, Hegele R A, Mitchell S J, Ransom T P P, Boctor D L, Spadafora P J, Jenkins A L, Mehling C, Relle L K, Connelly P W, Story J A, Furumoto, E J, Corey P, Wursch P: Effect on blood lipids of very high intakes of fibre in diets low in saturated fat and cholesterol. N Engl J. Med. 329:21-26, 1993

Jenkins D J A, Vuksan V, Wolever T M S, Ransom T P P, Vidgen E, Hegele R A, Leiter L, Josse R G, Abdolell, Patten R, Rao A V, Kendall C W C, Story, J A, Boctor D L, Corey P N: Diet and cardiovascular disease risk reduction: a place for fibre? Nutr Metab Cardiovasc Dis 5:251-259, 1995

Johnson C L, Rifkind B M, Sempos C T, Carroll M D, Bachorick P S, Briefel R R, Gordon D J, Burt V L, Brown C D, Lippel K, Cleeman J I: Declining serum total cholesterol levels among US adults: the National Examination Surveys. JAMA 269;3002-3008. 1993

Katona G, Aganovic I, Vuksan V, Skrabalo Z: The National Diabetes Programme in Malta: Final Report of Phases I and II. Geneva, World Health Organization, (NCD/OND/DIAB/83.2) 1983

Kuzuya T, Saito T, Yoshida S: Human C-peptide immunoreactivity (CPR) in blood and urine-Evaluation of radioimmunoassay method and its clinical applications. Diabetologia 12:511:518, 1976

Landin K, Holm G, Tengborn L, Smith U: Guar gum improves insulin sensitivity, blood lipids, blood pressure, and fibrinolysis in healthy men. Am J Clin Nutr 56:1061-1065, 1992

Liese A D, Mayer-Davis E J, Haffner S M: Development of the insulin resistance syndrome: An Epidemiologic Perspective. Epidemiol Rev 20:157-172, 1998

Livesey J H, Hodgkinson S C, Roud H R, Donald R A: Effect of time, temperature and freezing on the stability of immunoreactive LH, FSH, TSH, growth hormone, prolactin and insulin in plasma. Clin Biochem 13:151-157, 1980

Lloyd D, Marples J: Simple Calorimetry of glycated serum protein in a centrifugal analyzer. Clin Chem 30:1686-1688, 1984

McNamara J R, Schaefer E J: Automated enzymatic standardization lipid analyses for plasma and lipid fractions. Clin Chim Acta 166:108-111, 1987

Modan M, Halkin H, Almog S, Lusky A, Eshkol A, Shefi M, Shitrit A, Fuchs Z Hyperinsulinemia. A link between hypertension, obesity and glucose intolerance. J Clin Invest 75:809-817, 1985

Morgan L M, Tredger J A, Wright J, Marks V: The effect of soluble- and insoluble-fibre supplementation on post-prandial glucose tolerance, insulin and gastric inhibitory polypeptide secretion in healthy subjects. Br J Nutr 64:103-110, 1990

Montori V M, Farmer A, Wollan P C, Dinneen S F: Fish oil supplementation in Type 2 diabetes. Diabetes care 23;1407:1415, 2000

Mouratoff G J, Caroll N V, Scott E M: Diabetes mellitus in Eskimos. JAMA 199;107-122,1967

National Cholesterol Education Program: Second report of the expert panel on detection, evaluation, and treatment of high blood cholesterol in adults (adult treatment panel II). Circulation 89:1333-1445, 1994

Olson B H, Anderson S M, Becker M P, Anderson J W, Hunninghake D B, Jenkins D J, LaRosa J C, Rippe J M, Roberts D C, Story D B, Summerbell C D, Truswell A S, Wolever T M S, Morris D H, Fulgoni V L 3^{rd}: Psyllium-enriched cereals lower blood total cholesterol and LDL cholesterol, but not HDL cholesterol, in hypercholesterolemic adults: results of a meta-analysis. J Nutr 127:1973-1980, 1997

Prosky L. Asp N G, Furda I, DeVries J W, Schweizer T F, Harland B F: Determination of total dietary fibre in foods and food products: collaborative study. J Assoc Off Chem 68:677-679, 1985

Reaven G M (1994) Syndrome X: 6 years later. J Intern Med 736:13-22, 1994

Rimm E B, Ascherio A, Giovannucci E, Spiegelman D, Stampfer M J, Willett W C: Vegetable, fruit, and cereal fiber intake and coronary heart disease among men. JAMA 275:447451, 1996

Ridker P M: Novel risk factors for coronaray disease. Adv Intern Med 45;391:418, 2000

SAS Institute Inc: SAS/STAT User's guide. Version 6, 4th ed. Cary N C: SAS Institute Inc, 1989

Schaefer E J, Lichtenstein A H, Lamon-Fava S, Contois J H, Li Z, Rasmussen H, McNamara J R, Ordovas J M: Efficacy of a National Cholesterol Education Program Step 2 Diet in normolipidemic and hypercholesterolemic middle-aged men and elderly men and women. Arterioscler Thromb Vasc Biol 15:1079-1083, 1995

Salmeron J, Ascherio A, Rimm E B, Colditz G A, Spiegelman D, Jenkins D J, Stampfer M J, Wing A L, Willett W C: Dietary fiber, glycemic load, and risk of NIDDM in men. Diabetes Care 20:545-50, 1997

Savage P J: Cardiovascular complications of diabetes mellitus: what we know and what we need to know about prevention. Ann Intern Med 124:123-126, 1996

Stamler J, Vaccaro O, Neaton J D, Wentworth D. Diabetes, other risk factors, and 12 yr cardiovascular mortality for men in the multiple risk factor intervention trial. Diabetes Care 16:434-444,1993

Stefanick M L, Mackey S, Sheehan M, Ellsworth N, Haskell W L, Wood P D: Effects of diet and exercise in men and postmenopausal women with low levels of HDL cholesterol and high levels of LDL cholesterol. N Engl J Med 339:12-20,1998

Swain J F, Rouse I L, Curley C B, Sacks F M: Comparison of the effects of oat bran and low-fibre wheat on serum lipoprotein levels and blood pressure. N Engl J Med 322:147-152, 1990

The Agriculture Research Services. Composition of Foods, Agriculture Handbook No 8. Washington, D.C., US Department of Agriculture, 1992

The Lipid Research Clinics Population Studies Data Book. Vol. 2. The prevalence study-nutrient intake. Washington D.C.: Government printing office (NHI publication no. 82-2014), 1982

Thompson S G, Kienats J, Pyke S D M, Haverkate F, van de Loo J C W: Hemostatic factors and the risk of myocardial infarcton or sudden death N Engl J Med 332;635:641,1995

Trevisan M, Liu J, Bahsas F B, Menotti A: Syndrome X and mortality: A Population-based Study. Am J Epidemiol 148:958-966, 1998

Tuomilehto J, Silvasti M, Manninen V, Uusitupa M, Aro A: Guar gum and gemfibrozil-an effective combination in the treatment of hypercholesterolemia. Atherosclerosis 76:71-77, 1989

Tuomilehto J et. al. Diet, weight control and lifestyle reduce incidence in type 2 diabetes. N Engl J Med, 344;1343-41,2001

UK Prospective Diabetes Study (UKPDS) Group: Effect of intensive blood-glucose control with metformin on complications in overweight patients with type 2 diabetes: UKPDS 34. Lancet 352:854-865,1998

Warnick G R, Benderson J, Albers J J: Dextran sulfate-Mg⁺² precipitation procedure for quantitation of high-density lipoprotein cholesterol. Clin Chem 28: 1379-1388, 1982

Wei M, Gaskill S P, Heffner S M, Ster M P: Effects of diabetes and level of glycemia on all-cause and cardiovascular mortality: The San Antonio Heart Study. Diabetes Care 21(7):1167-1172, 1998

Wing M, Gaskill S P, Haffner S M, Stern M P: Effects of Diabetes and Level of Glycemia on All-Cause and Cardiovascular Mortality. Diabetes Care 21:1167-1172,1998

Wolever T M: Flaxseed and Glucose Metabolism. Flaxseed in Human Nutrition. Ed: Cunnane S C, Thompson L U. AOCS Press Champaigne, Ill., 157-164, 1995

World Health Organization Diabetes Mellitus: Report of the World Health Organization Study Group. Technical report No. 727:9-15, 1985

Wood P J: Physicochemical properties and physiological effects of the (1—-3)(1——4)-beta-D-glucan from oats. Adv Exp Med Biol 270:119-27, 1990

### REFERENCES ON CHIA

Hernandez Gomez, J. A. 1994. Chia (Salvia hispanica): Antecedentes y perspectivas en México, 173-180 in I Simposium internacional sobre Etnobotanica en Mesoamerica, Universidad Autónoma Chapingo.

Bukasov, S. M. 1963 Las plantas cultivadas de México, Guatemala y Colombia. Publicación miscelánea N° 20. Instituto Interamericano de Ciencias Agrícolas de la OEA. Zona andina, Lima, Perú pp.193-194

Giller, H. 1981 Le Chia, graine mucilagineuse mexicaine, fait son apparition en France” Journal d'Agricultire Traditionelle et de Botanique Appliquee 28(2): 183-187

Mann, P. 1959 Systematics of Flowering Plants. Reimpreso Methuen and Co. LTD London pp 201-204

Martínez, M. 1923 Catálogo alfabe'tico de nombres vulgares y cientificos de plantas que existen en México. Secretaría de Agricultura y Fomento. Dirección de Estudios Biológicos. México

Martínez, M 1959 Plantas útiles de la flora mexicana. Ed. Botas

Rulfo J. M. 1937. La Chia. Agricultura (México) 1:28-37

Urbina M. 1983. La chia y sus aplicaciones. In Revista de Geografía Agrícola. Análisis regional de la agricultura 4:123-133

Flowers, H. 1882 Chia seed. Amer. Jour. Pharm Tomo L I V, Pag 227-229

Starr, G. 1985. New world Dalvias for cultivation in Southern Arizona. Desert Plantas 7(4): 167-193

Brown, J H. 1997 The rediscovery of Chia, a Nutritious Grain of Mesoamerica. International Flora Technologies LTD.

Anderson A. J. O. and Dibble, C. E. An Ethnobiography of the Nahuatl. The Florentine Codex, (translation of the work by Fr. Bernardino Sahagun), Books 10-11, from the period 1558-1569

Orozco de Rosas, G. Chia (Salvia hispanica L.) Una alternativa a los cultivos tradicionales. Fundación Produce Jalisco

Miranda, C. S. 1978 Evolución de cultivares nativos de México. Ciencia y Desarrollo 3:21. pp 130.131

Urbina, M. La chia y sus aplicaciones. Geografla Agricola 4. Universidad Autónoma Chapingo

Scheer, James; “Magic of Chia: Revival of an Ancient Wonder Food”, (North Atlantic Books, Frog Ltd, 2001)

What is claimed is:

1. The use of an effective amount of chia seed in one or more of the following uses:
(a) the treatment and/or management of diabetes; and/or
(b) the treatment and/or management of diabetes associated conditions or risk factors;
(c) the treatment and/or management of cardiovascular disease or associated rick factors.

2. The use of claim 1 wherein the associated conditions or risk factors are selected from the group consisting of one or more of the following: high blood pressure, high blood glucose levels, post-prandial glycemia, inflammatory factors, coagulation, fibrinolytic factors, iron status and endothelial function.

3. The use of claim 2 wherein the associated condition or risk factor is an inflammatory factor, C-reactive protein.

4. The use of claim 2 wherein the associated condition or risk factor is coagulation indicated by high fibringogen and/or factor VIII levels, and/or vonWillebrand factor.

5. The use of claim 2 wherein the associated condition or risk factor is a fibrinolytic factor, t-PA.

6. The use of claim 2 wherein the associated condition or risk factor is low iron status, indicated by feritinin levels.

7. The use of claim 2 wherein the associated condition or risk factor is reduced endothelial function, indicated by nitric oxide levels.

8. The use of any one of claims 1-7, wherein the effective amount of chia seed is about 5-100 g/day.

9. The use of anyone of claims 1-8, wherein the chia seed is in a whole seed, ground powder or liquid.

10. The use of anyone of claims 1-8 wherein the chia seed is administered in the form of a chia seed composition.

11. The use of anyone of claims 1-8 wherein the chia seed is a chia seed-like composition.

12. The use of an effective amount of chia seed or a chia seed composition to:
(a) reduce blood glucose levels;
(b) reduce fibrinogen, factor VIII and/or vWLBR factor;
(c) increase t-PA levels;
(d) reduce CRP levels:
(e) increase feritinin levels:
(f) reduce fasting and postprandial glucose levels
(g) reduce nitric oxide level;
(h) reduce systolic blood pressure levels:
(i) reduce diastolic blood pressure levels:

13. The use of an effective amount of chia seed, a chia seed composition and/or a chia seed-like compositions to: control or manage, or treat any conditions or reduce risk of any development of any condition associated with any one or more of the indicators listed in (a)-(i) of claim 12.

14. The use of claim 13 wherein he conditions are selected from the group consisting of: glycemia, diabetes, cardiovascular disease, inflammation, fibrinolysis, coagulation, endothelial function, thrombosis, arrhythmia, platelet aggregation, atherosclerosis and iron status.

15. The use of an effective amount of Salvia hispanica L. (Chia) for the treatment of diabetes in an animal in need thereof.

16. The use of claim 15 wherein the effective amount is administered alone or with the meal in order to improve diabetes control in the animal.

17. The use of anyone of claims 1-16 wherein the effective amount is the equivalent of 5 to 100 grams of chia seed per day).

18. The use of claim 17 wherein the effective amount is administered orally.

19. The use according to anyone of claims 1-17 wherein the effective amount is administered either prior, after to a meal or during the meal.

20. The use according to claim 19 wherein the effective amount is administered by seeds alone, in a liquid, a powder, or as a part of a food product, or beverage.

21. The use of anyone of claims 1-20 for the treatment of long term diabetes, heart disease, or oxidative stress

22. The use of claim 1-20 to reduce progression of the animal's thrombo-athersoclerotic process.

23. The use of claim 22 wherein the animal's thrombo-athersoclerotic process is reduced by reducing inflammation or improving coagulation in an animal.

24. A use of an effective amount of chia seed, chia seed composition or a chia seed-like composition for treating hypertension in type 2 diabetes individuals.

25. A use of an effective amount of chia seed, chia seed composition or chia seed-like composition for increasing nitric oxide to improve endothelial function

26. A use of an effective amount of chia seed, chia seed composition or chia seed-like composition for increasing serum feritinin to improve iron status.

27. A composition of matter for reducing blood glucose comprising Saliva Hispanica L. a sufficient amount of which when given to an animal at an appropriate time reduces blood glucose in the animal.

28. A composition of matter for reducing blood glucose comprising an extract of Saliva Hispanica L. a sufficient amount of which when given to an animal at an appropriate time reduces blood glucose in the animal.

29. The composition according to any one of claims 16-18 wherein the composition is formulated into a liquid, powder or formulated as part of a food.

30. A method for reducing blood glucose in an animal comprising administering to the animal a sufficient amount of Saliva Hispanica L. at an appropriate time in order to reduce blood glucose in the animal.

31. A method for reducing blood glucose in an animal comprising administering to the animal a sufficient amount of an extract of Saliva Hispanica L. at an appropriate time in order to reduce blood glucose in the animal.

32. A method according to claim 30 or 31 wherein the Saliva Hispanica L. or extract is administered before a meal or with a meal.

33. A method according to claim 32 wherein administration before meal occurs from about 1 to about 180 minutes before the meal.

34. A method according to any one of claims 30-23 wherein the composition is administered as a food, a powder, or a liquid.

35. A method for the treatment or lowering the risks of long term diabetes or heart disease, comprising anyone of the methods of claims 30-34.

36. A method of treating type 2 diabetes comprising any one of the methods according to claims 30-35.

37. A method for reducing systolic blood pressure or diastolic blood pressure, comprising a method according to any one or claims 30-35.

38. A method for doing one or more of the following:
(a) reduce blood glucose levels;
(b) reduce fibrinogen, factor VIII and/or vWLBR factor;
(c) increase t-PA levels;
(d) reduce CRP levels:
(e) increase feritinin levels:
(f) reduce fasting glucose levels
(g) reduce nitric oxide levels;
(h) reduce systolic blood pressure levels:
(i) reduce diastolic blood pressure levels:
by administering to a patient in need thereof an effective amount of chia seed, a chia seed composition and/or a chia seed-like compositions

39. The method of claim 38 wherein the effective amount of chia seed, a chia seed composition and/or a chia seed-like compositions is administered to a patient in need thereof to: control or manage or treat any conditions or reduce risk of the development of any conditions associated with any one or more of the indicators listed in (a)-(i) of claim 38.

40. The method of claim 38 wherein the conditions are selected from the group consisting of: glycemia, diabetes, cardiovascular disease, inflammation, fibrinolysis, coagulation, endothelial function, thrombosis, arrhythmia, platelet aggregation, atherosclerosis, and iron status.
